(19) United States
(12) Patent Application Publication
(10) Pub. No.: US 2026/0174427 A1
(43) Pub. Date: Jun. 25, 2026

(54) DEVICE FOR SENSING SURGICAL SUTURE

(71) Applicant: METAL INDUSTRIES RESEARCH & DEVELOPMENT CENTRE, Kaohsiung (TW)

(72) Inventors: Kai-Szu Lo, Tainan City (TW); Ming-Hui Chen, Kaohsiung City (TW)

(21) Appl. No.: 18/987,111

(22) Filed: Dec. 19, 2024

(51) Int. Cl. A61B 17/04

(52) U.S. Cl. CPC A61B 17/0469; A61B 2017/0496; A61B 2562/00

(57) ABSTRACT

A device for sensing a surgical suture can be implanted on a biological tissue, e.g. bone, to sense the tension applied to the biological tissue by the surgical suture. The device includes a carrier, a wireless sensor and a positioning pin. The wireless sensor is mounted on a carrier body of the carrier. A second connection portion of the positioning pin is movably pivoted to a first connection portion of the carrier. Thus, the wireless sensor can be rotated and/or swung with the carrier relative to the positioning pin to sense the surgical suture tension.

### FIELD OF THE INVENTION

This invention relates to a device for sensing surgical suture, which is used to sense a surgical suture tension during or after surgery.

### BACKGROUND OF THE INVENTION

Tendon suture and bone fixation surgery using surgical suture are common medical treatments. However, it is unable to know whether the surgical suture tension is proper during surgery operation, and unable to know whether the surgical suture tension is changed after the surgery. For this reason, the surgery's intended effect was achieved or not is unknown.

### SUMMARY OF THE INVENTION

One object of the present invention is to provide a device for sensing surgical suture tension during or after surgery.

A device for sensing surgical suture includes a carrier, a wireless sensor and a positioning pin. The carrier includes a carrier body and a first connection portion. The wireless sensor is mounted on the carrier body and provided for sensing a surgical suture's tension. The positioning pin includes a pin body and a second connection portion, the second connection portion is movably pivoted to the first connection portion of the carrier so the wireless sensor can be rotated and/or swung with the carrier relative to the positioning pin.

The second connection portion of the positioning pin is movably pivoted to the first connection portion of the carrier, thus, the wireless sensor mounted on the carrier can be rotated and/or swung with the carrier relative to the positioning pin for sensing the surgical suture tension.

### BRIEF DESCRIPTION OF DRAWINGS

FIG. 1 is a perspective assembly diagram illustrating a device for sensing surgical suture in accordance with one embodiment of the present invention.

FIG. 2 is a perspective exploded diagram illustrating a device for sensing surgical suture in accordance with one embodiment of the present invention.

FIG. 3 is a perspective assembly diagram illustrating a device for sensing surgical suture in accordance with one embodiment of the present invention.

FIG. 4 is a perspective exploded diagram illustrating a device for sensing surgical suture in accordance with one embodiment of the present invention.

FIG. 5 is a cross-section view diagram illustrating a device for sensing surgical suture in accordance with one embodiment of the present invention.

FIG. 6 is a cross-section view diagram illustrating a device for sensing surgical suture in accordance with one embodiment of the present invention.

FIG. 7 is a top view diagram illustrating a device for sensing surgical suture in accordance with one embodiment of the present invention.

### DETAILED DESCRIPTION OF THE INVENTION

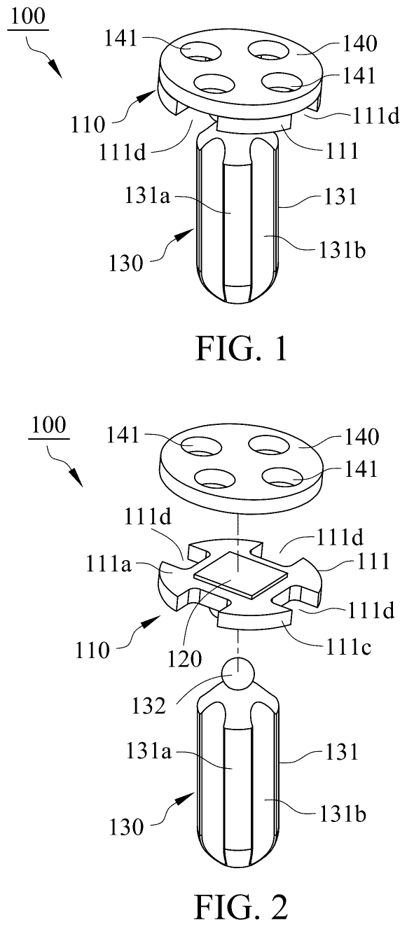

FIGS. 1 to 4 illustrate a device 100 for sensing surgical suture in accordance with one embodiment of the present invention. The device 100 is provided to be implanted on a biological tissue (not shown, e.g. bone) and to sense tension of a surgical suture L applied to the biological tissue.

With reference to FIGS. 1 to 4, the device 100 includes a carrier 110, a wireless sensor 120 and a positioning pin 130. The wireless sensor 120, which may be a passive pressure sensor, is mounted on the carrier 110 and provided to sense the tension applied to the biological tissue by the surgical suture L. The positioning pin 130 is movably pivoted to the carrier 110.

As shown in FIGS. 1 to 5, the carrier 110 included a carrier body 111 and a first connection portion 112. The wireless sensor 120 is mounted on the carrier body 111, and in this embodiment, the wireless sensor 120 is mounted on a first surface 111a of the carrier body 111 and it protrudes from the first surface 111a. The first connection portion 112 is located on a second surface 111b of the carrier body 111 and it protrudes from the second surface 111b. The first connection portion 112 may be a universal ball seat or a ball joint. Preferably, multiple restriction holes 111d are provided on the carrier body 111 and they are recessed on an edge 111c of the carrier body 111.

With reference to FIGS. 1 to 5, the positioning pin 130 includes a pin body 131 and a second connection portion 132 which is located on one end of the pin body 131. The positioning pin 130 is movably pivoted to the first connection portion 112 of the carrier 110 via its second connection portion 132, thus the carrier 110 can be rotated and/or swung around the first connection portion 112 or the second connection portion 132 relative to the positioning pin 130. Likewise, the wireless sensor 120 mounted on the carrier 110 can be rotated and/or swung with the carrier 110 relative to the positioning pin 130. The second connection portion 132 may be a universal ball seat or ball joint. In this embodiment, the first connection portion 112 of the carrier 110 is a ball seat, and the second connection portion 132 of the positioning pin 130 is a ball joint. And in other embodiments, the first connection portion 112 of the carrier 110 is a ball joint, and the second connection portion 132 of the positioning pin 130 is a ball seat. The ball joint is designed to be pivoted to the ball seat such that the wireless sensor 120 can be rotated and/or swung with the carrier 110 relative to the positioning pin 130. Preferably, there are multiple restriction grooves 131b provided on a surface 131a of the pin body 131.

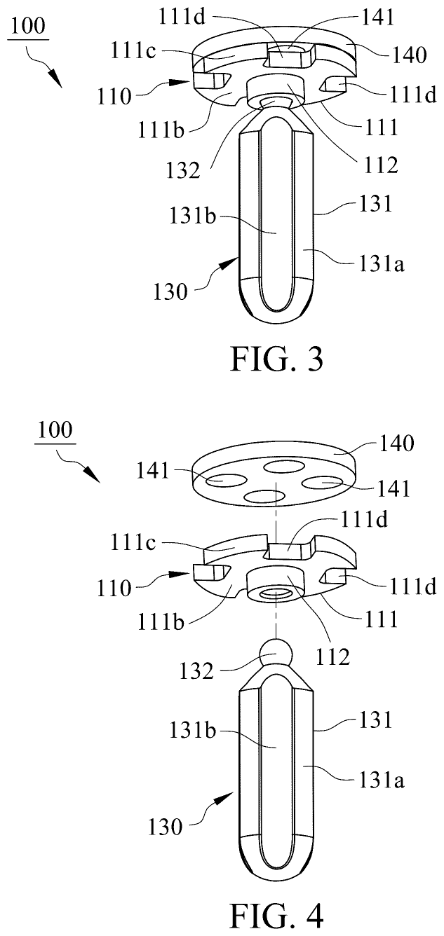

With reference to FIG. 3, the device 100 is implanted on the biological tissue by the positioning pin 130 during a surgery of stitching the biological tissue (e.g. tendon) or fixation of the biological tissue (e.g. bones). While the positioning pin 130 is inserted into a positioning hole (not shown) on the biological tissue and the positioning hole or the position pin 130 is oblique, the carrier 110 can be rotated and/or swung around the first connection portion 112 or the second connection portion 132 according to the direction of the surgical suture L inserting into or through the biological tissue owing to the second connection portion 132 is movably pivoted to the first connection portion 112, and the wireless sensor 120 can be rotated and/or swung relative to the positioning pin 130 with the carrier 110 and be pressed by the surgical suture L precisely to sense the tension of the surgical suture L. Furthermore, the surgical suture L can be restricted by the restriction holes 111d on the carrier body 111 and/or the restriction grooves 131b on the pin body 131 so the surgeon can tie the surgical suture L easily. The surgical suture L restricted by the restriction holes 111d on the carrier body 111 and/or the restriction grooves 131b on the pin body 131 will not move or separate from the wireless sensor 120 so the wireless sensor 120 can sense the tension of the surgical suture L precisely during or after the surgery.

With reference to FIGS. 1 to 4 and 6, the device 100 further includes a pressing element 140 in another embodiment. After inserting the surgical suture L through the pressing element 140, the surgical suture L presses the pressing element 140 to allow the pressing element 140 to press the wireless sensor 120 such that the wireless sensor 120 can sense the tension of the surgical suture L.

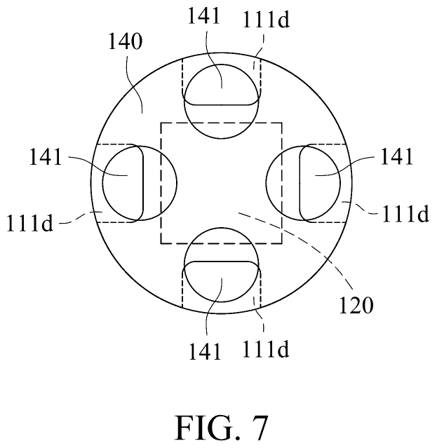

With reference to FIGS. 1 to 4, 6 and 7, there are multiple holes 141 on the pressing element 140, and at least one hole 141 is aligned with one of the restriction holes 111d on the carrier body 111. After inserting the surgical suture L into or through the biological tissue, the surgical suture L is inserted through the holes 141 to press the pressing element 140. Consequently, the pressing element 140 can press the wireless sensor 120 to allow the wireless sensor 120 to sense the tension of the surgical suture L.

With reference to FIGS. 1 to 4 and 6, because the second connection portion 132 of the positioning pin 130 is movably pivoted to the first connection portion 112 of the carrier 110, the carrier 110 can be rotated and/or swung around the first connection portion 112 or the second connection portion 132 based on the direction of the surgical suture L inserting into or through the biological tissue, and the wireless sensor 120 can be rotated and/or swung relative to the positioning pin 130 with the carrier 110. As a result, the surgical suture L can press the pressing element 140 precisely and allow the pressing element 140 to press the wireless sensor 120 precisely.

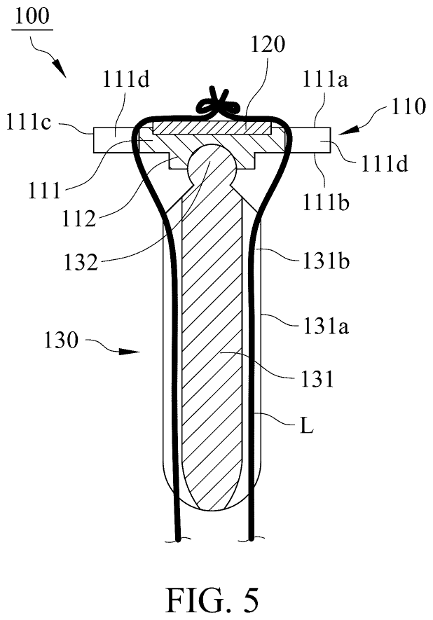

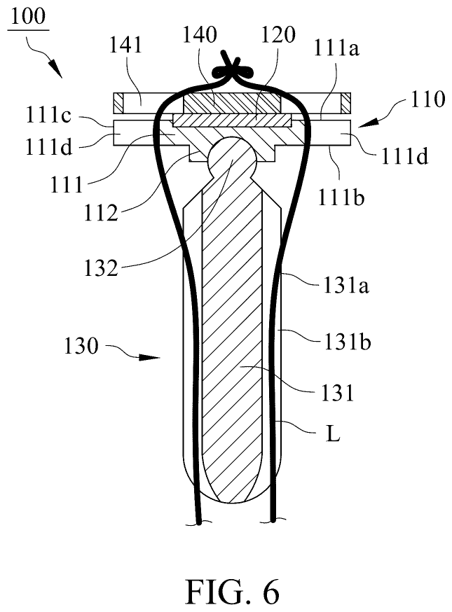

In the present invention, the second connection portion 132 of the positioning pin 130 is designed to be movably pivoted to the first connection portion 112 of the carrier 110, so the wireless sensor 120 can be rotated and/or swung relative to the positioning pin 130 with the carrier 110. The surgical suture L can press the wireless sensor 120 (as shown in FIG. 5) or press the pressing element 140 and allow the pressing element 140 to press the wireless sensor 120 (as shown in FIG. 6), thus, the wireless sensor 120 can precisely sense the tension applied to the biological tissue by the surgical suture L.

While this invention has been particularly illustrated and described in detail with respect to the preferred embodiments thereof, it will be clearly understood by those skilled in the art that is not limited to the specific features shown and described and various modified and changed in form and details may be made without departing from the scope of the claims.

1. A device for sensing surgical suture comprising:
a carrier including a carrier body and a first connection portion;
a wireless sensor mounted on the carrier body and configured to sense a surgical suture tension; and
a positioning pin including a pin body and a second connection portion, wherein the second connection portion is movably pivoted to the first connection portion such that the wireless sensor is configured to be rotated and/or swung with the carrier relative to the positioning pin.

2. The device in accordance with claim 1, wherein the wireless sensor is configured to be pressed by the surgical suture.

3. The device in accordance with claim 1 further comprising a pressing element, wherein the pressing element is configured to be pressed by the surgical suture and configured to press the wireless sensor.

4. The device in accordance with claim 3, wherein a plurality of holes are provided on the pressing element, the plurality of holes are configured for insertion of the surgical suture to allow the pressing element to press the wireless sensor.

5. The device in accordance with claim 1, wherein the wireless sensor protrudes from a first surface of the carrier body.

6. The device in accordance with claim 1, wherein the first connection portion is a ball seat, the second connection portion is a ball joint configured to be pivoted to the ball seat.

7. The device in accordance with claim 1, wherein a plurality of restriction holes are provided on the carrier body and recessed on an edge of the carrier body.

8. The device in accordance with claim 4, wherein a plurality of restriction holes are provided on the carrier body and recessed on an edge of the carrier body, at least one of the plurality of holes on the pressing element is aligned with one of the plurality of restriction holes on the carrier body.

9. The device in accordance with claim 1, wherein a plurality of restriction grooves are provided on a surface of the pin body and configured to restrict the surgical suture.

10. The device in accordance with claim 1, wherein the first connection portion is located on a second surface of the carrier body.
